(19) United States
(12) Patent Application Publication
(10) Pub. No.: US 2013/0018277 A1
(43) Pub. Date: Jan. 17, 2013

(54) Non-invasive intracranial pressure monitor

(71) Applicant: Jung-Tung Liu, Taichung (TW)

(72) Inventor: Jung-Tung Liu, Taichung (TW)

(21) Appl. No.: 13/183,460

(22) Filed: Jul. 15, 2011

(51) Int. Cl. A61B 5/00

(52) U.S. Cl. 600/561

(57) ABSTRACT

A non-invasive intracranial pressure monitor includes an annular flexible headband member comprising a crossing arcuate rail member and a hook and loop fastener having a fastener portion at one end of the headband member and a cooperating fastener portion at the other end of the headband member; and a monitoring assembly comprising an inverted T-shaped, hollow seat slidably mounted on the rail member, a fastener driven through two ends of the seat for retaining the seat on the rail member, a receptacle extending downward from the seat opposing the fastener, a biasing member disposed in the receptacle, and a microsensor having one end secured to the biasing member and the other end moveably projecting out of the receptacle.

### BACKGROUND OF THE INVENTION

1. Field of the Invention

The invention relates to apparatuses of measuring intracranial pressure and more particularly to a piezoresistor based non-invasive intracranial pressure monitor.

2. Description of Related Art

Intracranial pressure (ICP) of an individual may increase suddenly due to brain swelling, obstruction of cerebral spinal fluid passageways, or brain bleeding because of traffic accident. The intracranial hypertension may painfully press blood vessels and nerves within the brain. This is a life threatening event. Doctors specializing in neurosurgery are aware of the danger and thus many drugs and surgery techniques have been proposed to effectively decrease ICP. The criticality of dispensing such drug to a patient and/or performing a surgery to the patient is that an accurate ICP must be known prior to any treatment.

Various apparatuses and methods for ICP measurement are known in the art. For example, one technique involves inserting a catheter through skull into a cerebral spinal fluid passageway for sucking fluid for analysis. Such implantation is invasive and has danger of infection, patient discomfort, and other adverse effects.

Taiwan Patent Number 1318,569 discloses a method of for measuring ICP by comparing agents by transmitting ultrasonic waves into brain. In detail, the method comprises transmitting an ultrasonic wave signal of band width toward a target in the brain, receiving a signal reflecting from micro bubbles filled with comparison agents in the target of the brain, analyzing spectrum of the reflecting signal to obtain a low frequency response having a band width substantially equal to that of the transmitting signal, calculating a resonance frequency of the micro bubbles based on band width and strength of the low frequency response, calculating sizes of the micro bubbles based on the resonance frequency and properties of the comparison agents, and obtaining an ICP of the target by performing a calculation.

U.S. Pat. No. 4,026,276 discloses a pressure monitoring apparatus implantable in the cranium to measure intracranial pressure. The apparatus comprises a passive resonant circuit having a natural frequency influenced by ambient pressure. The resonant circuit has inductance and capacitance capability for comparing the local environmental pressure to that of a volume of gas trapped inside the apparatus, the environmental pressure being measured by observation of the frequency at which energy is absorbed from an imposed magnetic field located externally of the cranium.

While above patents are directed to non-invasive apparatus and methods, they are disadvantageous due to complicated components, inconvenience in use, inaccurate positioning of the target in the brain, low utility in diagnosis, and high cost.

Notwithstanding the prior art, the invention is neither taught nor rendered obvious thereby.

### SUMMARY OF THE INVENTION

It is therefore one object of the invention to provide a non-invasive apparatus for sensing intracranial pressure comprising an annular flexible headband member comprising a crossing arcuate rail member and a hook and loop fastener having a fastener portion at one end of the headband member and a cooperating fastener portion at the other end of the headband member; and a monitoring assembly comprising an inverted T-shaped, hollow seat slidably mounted on the rail member, a fastener driven through two ends of the seat for retaining the seat on the rail member, a receptacle extending downward from the seat opposing the fastener, a biasing member disposed in the receptacle, and a microsensor having one end secured to the biasing member and the other end moveably projecting out of the receptacle.

The above and other objects, features and advantages of the invention will become apparent from the following detailed description taken with the accompanying drawings.

### BRIEF DESCRIPTION OF THE DRAWINGS

FIG. 1 is a perspective view of a non-invasive intracranial pressure monitor according to the invention;

FIG. 2 is a side elevation of the intracranial pressure monitor put on the head of a patient;

FIG. 3 is a longitudinal sectional view of the intracranial pressure monitor and adjacent portion of the head in FIG. 2;

FIG. 4 is a longitudinal sectional view of the microsensor and adjacent portion of the head in FIG. 3 showing the measurement of intracranial pressure;

FIG. 5 is a perspective view of the intracranial pressure monitor put on the head showing an initial adjustment of the intracranial pressure monitor on the head; and

FIG. 6 is a view similar to FIG. 5 showing a precise adjustment of the intracranial pressure monitor on the head by sliding the seat along the rail member.

### DETAILED DESCRIPTION OF THE INVENTION

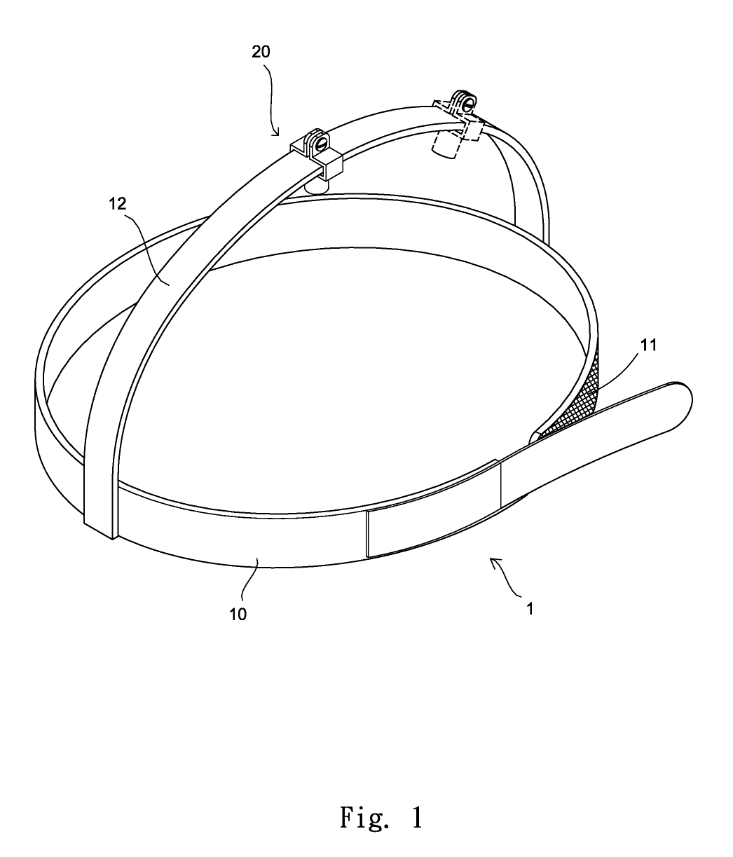

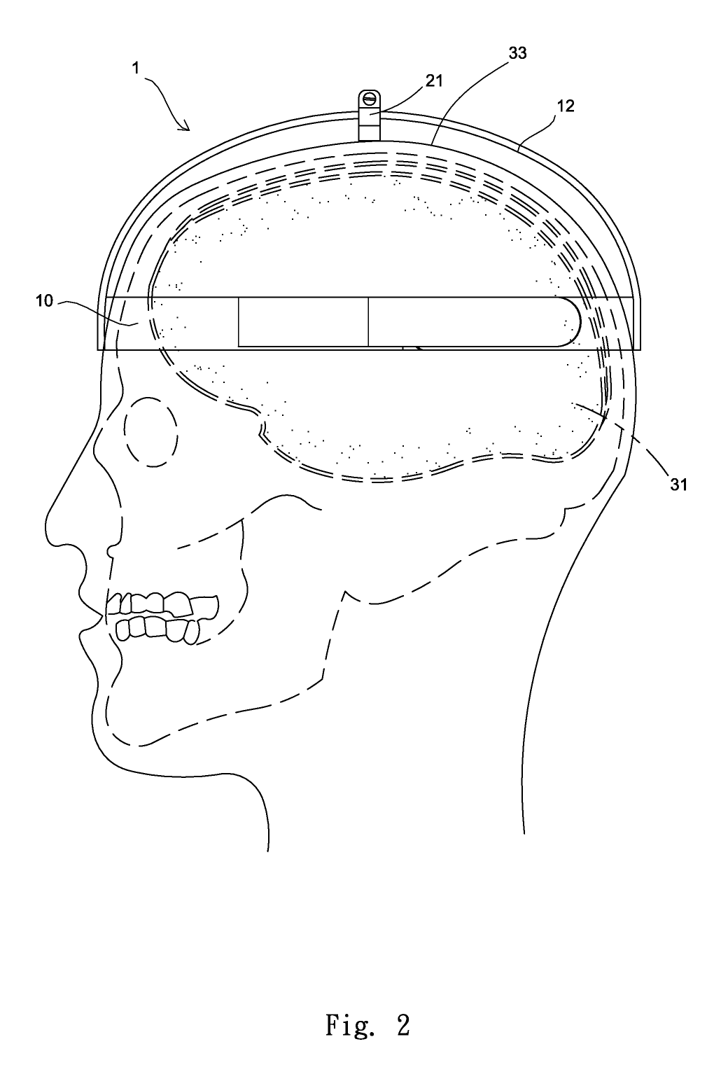

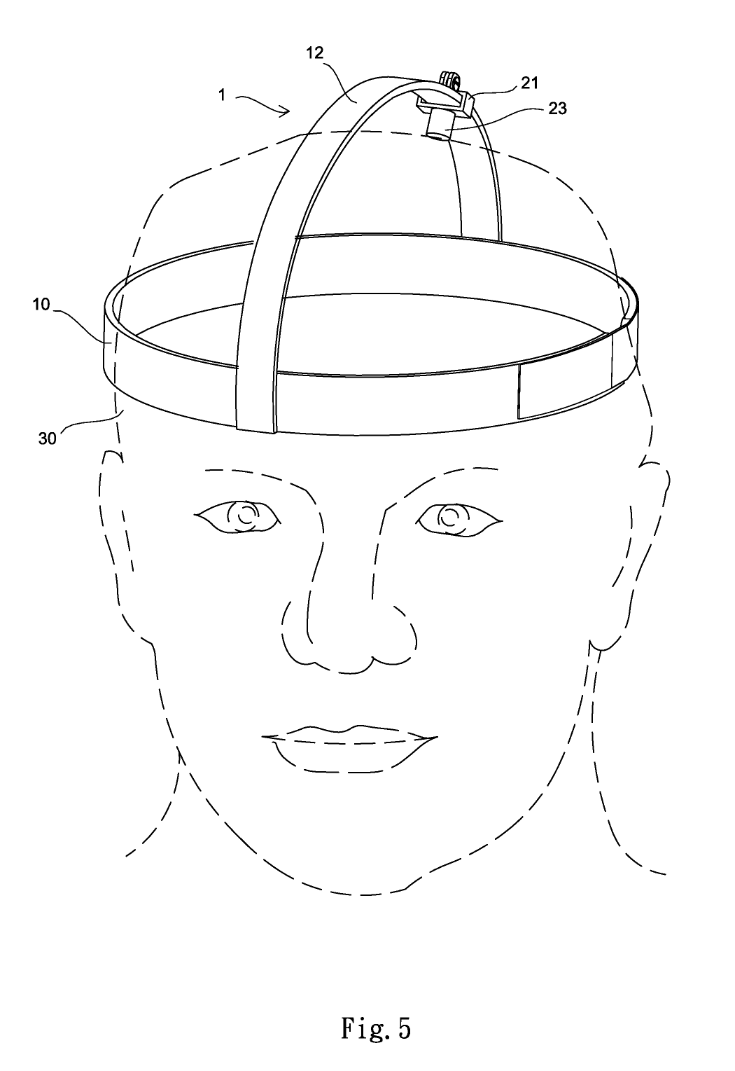

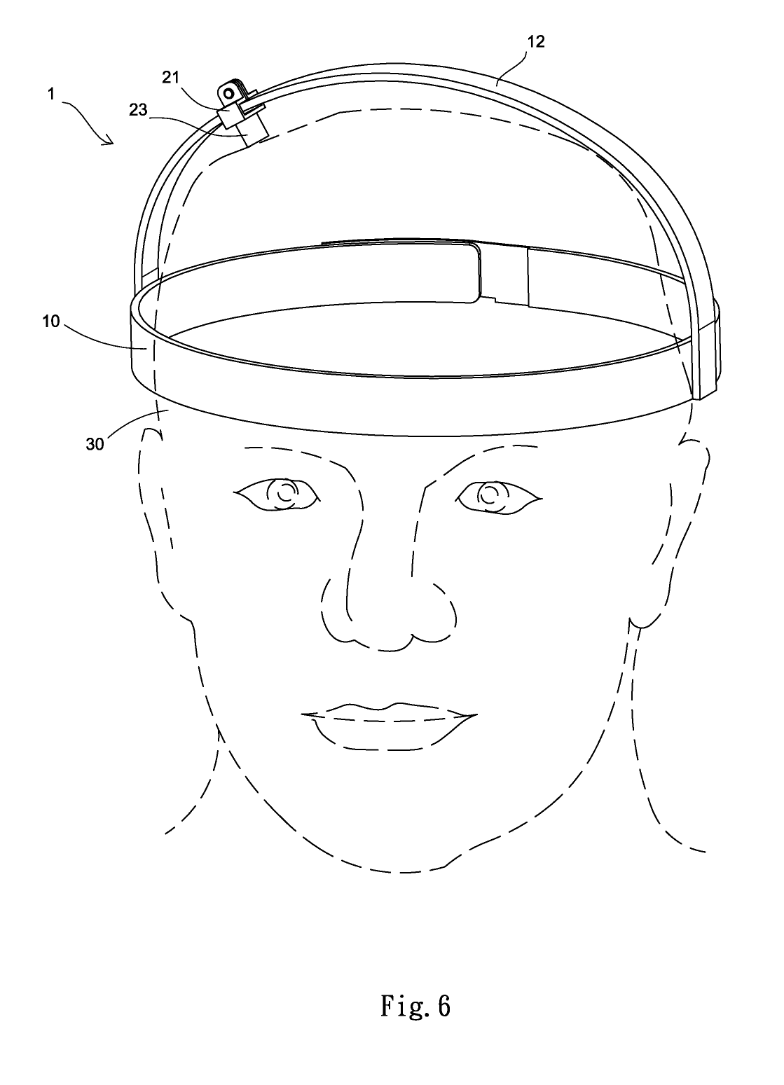

Referring to FIGS. 1 to 6, a non-invasive intracranial pressure monitor 1 in accordance with the invention is shown. The monitor 1 is shaped as a head gear and comprises the following components as discussed in detail below.

An annular headband member 10 is formed of flexible material (e.g., plastic or metal) and comprises a Velcro fastener 11 formed on both ends, and an arcuate rail member 12 crossing portions of the headband member 10 other than both ends. A monitoring assembly 20 comprises an inverted T-shaped, hollow seat 21 slidably mounted on the rail member 12, a fastener (e.g., screw) 22 driven through two ends of the seat 21 for retaining the seat 21 on the rail member 12, a short cylindrical receptacle 23 extending downward from the bottom of the seat 21, a torsion spring 24 disposed in the receptacle 23, and a microsensor 25 having one end secured to the torsion spring 24 and the other end projecting out of the receptacle 23 due to the expansion of the torsion spring 24. That is, the microsensor 25 is a spring depressible member.

The microsensor 25 is electrically connected to an external processor (not shown) and is a piezoresistive pressure sensor. The microsensor 25 comprises a membrane 251, a flexible substrate 252 formed of plastic, the substrate 252 being disposed on the top of the membrane 251, a cavity 253 in the substrate 252 communicating with the membrane 251, and a plurality of (e.g., two piezoresistors) 254 formed on the bottom of the membrane 251. Pressure can deflect the membrane 251 and the deflection is proportional to pressure. Further, resistance of the piezoresistor 254 may change in proportional to the deflection as detailed later. The provision of a plurality of piezoresistors 254 can increase accuracy of the pressure measured by the microsensor 25.

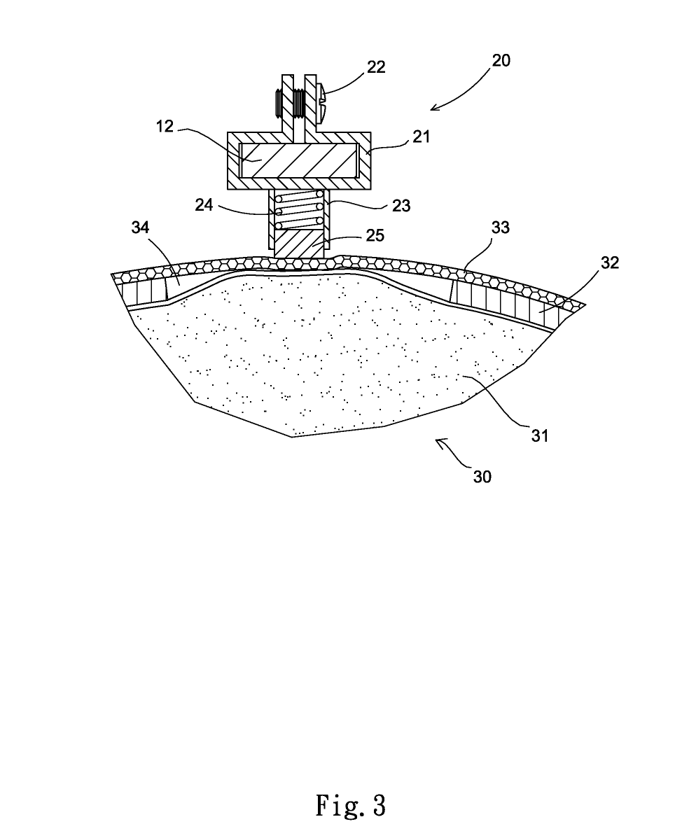

As shown in FIG. 3, a human head 30 comprises, from inner portion to outer portion, a brain 31, a skull 32, and a scalp 33. The brain 31 may be injured due to traffic accident or the brain 31 may be swelled due to disease. Thus, a hole 34 may be formed in the skull 32 due to breakage or swelling. The brain 31 thus is in close proximity to the scalp 33 through the hole 34. Fortunately, there is no contact of the brain 31 and the external. Otherwise, it may cause infection to the brain 31.

As shown in FIGS. 3 to 6, the headband member 10 is firstly put on the head 30 of a patient. Next, a medical employee may adjust and fasten the Velcro fastener 11 to accommodate the size of the head 30, i.e., adjusting positioning of the headband member 10 along Z-axis. Next, the medical employee may slide the monitoring assembly 20 along the rail member 12 until a target is reached, i.e., adjusting positioning of the monitoring assembly 20 on a Cartesian coordinate (i.e., (X-Y) plane). The torsion spring 24 may expand or compress in response to the microsensor 25 gently sliding on the slightly irregular contour of the scalp 33. The adjustment finishes when the microsensor 25 is disposed in alignment with the hole 34 in the skull 32. Thereafter, the medical employee may tighten the screw 22 to secure the monitoring assembly 20 and the rail member 12 together. Finally, the medical employee may activate the microsensor 25 to begin pressure measurement (i.e., measuring ICP).

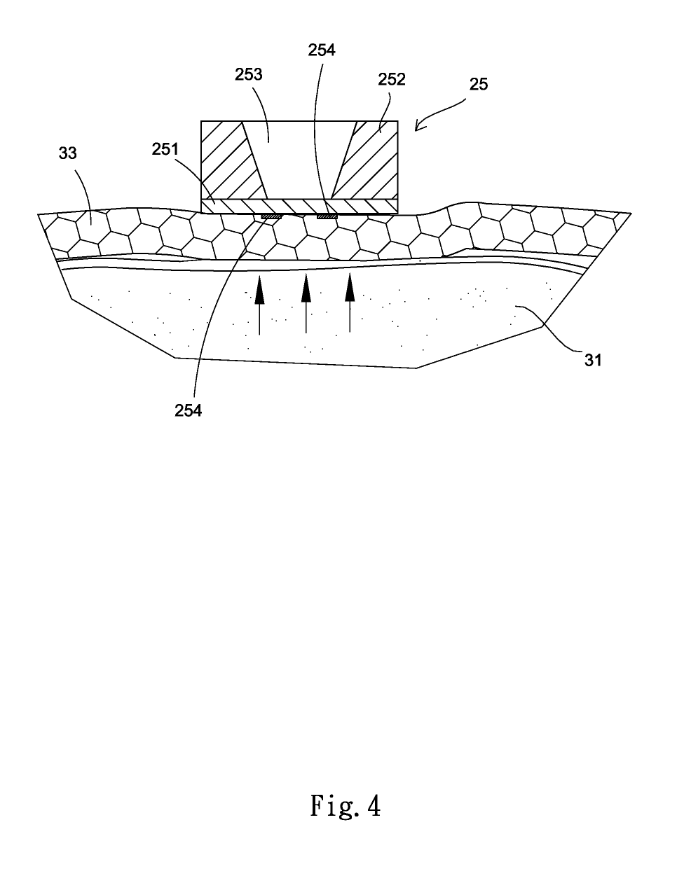

Operation of the microsensor 25 will be described in detail below. Pressure of the target (i.e., injured portion of the brain 31 or diseased portion thereof) may increase greatly. The pressure deflects the membrane 251 by applying through the hole 34 and the scalp 33 and the deflection is proportional to the pressure (see FIG. 4). Further, resistance of the piezoresistor 254 may change in proportional to the deflection. The resistance change can be measured with a Wheatstone bridge. The measured resistance change is converted into a corresponding ICP which is in the form of electrical signal sent to the processor for further processing. Advantageously, the plurality of piezoresistors 254 can increase accuracy of the pressure measured by the microsensor 25 (i.e., being very sensitive and accurate).

Alternatively, the monitoring assembly 20 (i.e., the spring depressible microsensor 25) can be replaced with an ultrasonic based monitor in another embodiment. In detail, the ultrasonic based monitor comprises a transmission module for transmitting an ultrasonic wave signal of band width toward a target in the injured or diseased portion of the brain, the signal being a short pulse signal, a receiving module for receiving a signal reflecting from the target in the brain, the reflecting signal being very accurate due to minimum decay, and a sending module for sending the reflecting signal to an external processor. The processor can analyze spectrum of the reflecting signal to obtain a base frequency response, a first resonance response, a second resonance response, and a low frequency response. A resonance frequency can be obtained by analyzing and calculating the above responses. Finally, an accurate ICP of the target can be measured by performing a calculation with respect to the resonance frequency.

The invention has the following advantages: Non-invasive. No injury to the brain. No infection to the brain. Accurate ICP measurement due to precise positioning of the microsensor by both Velcro fastener and spring based adjustments.

While the invention has been described in terms of preferred embodiments, those skilled in the art will recognize that the invention can be practiced with modifications within the spirit and scope of the appended claims.

What is claimed is:

1. A non-invasive apparatus for sensing intracranial pressure comprising:
an annular flexible headband member comprising a crossing arcuate rail member and a hook and loop fastener having a fastener portion at one end of the headband member and a cooperating fastener portion at the other end of the headband member; and
a monitoring assembly comprising an inverted T-shaped, hollow seat slidably mounted on the rail member, a fastener driven through two ends of the seat for retaining the seat on the rail member, a receptacle extending downward from the seat opposing the fastener, a biasing member disposed in the receptacle, and a microsensor having one end secured to the biasing member and the other end moveably projecting out of the receptacle.

2. The non-invasive apparatus for sensing intracranial pressure of claim 1, wherein the headband member is put on the head of an individual, the hook and loop fastener is pressed to fasten after adjusting positioning of the headband member along Z-axis, the monitoring assembly is slid along the rail member for adjusting positioning of the monitoring assembly on a Cartesian coordinate so that the biasing member expands or compresses in response to the microsensor sliding on the head, and the positioning adjustment of the monitoring assembly on the Cartesian coordinate finishes when the microsensor is disposed in alignment with a target in the head.

3. The non-invasive apparatus for sensing intracranial pressure of claim 2, wherein the microsensor is a piezoresistive pressure sensor and comprises a membrane, a flexible substrate disposed on the top of the membrane, a cavity in the substrate communicating with the membrane, and a plurality of piezoresistors formed on the bottom of the membrane, wherein pressure from the target deflects the membrane, and wherein resistance of each of the piezoresistors changes in proportional to the deflection of the membrane.

4. The non-invasive apparatus for sensing intracranial pressure of claim 3, wherein the pressure from the target deflects the membrane by applying through a hole in the head and the scalp of the head, the deflection of the membrane is proportional to the pressure, the resistance of each of the piezoresistors changes in proportional to the deflection of the membrane, the resistance change is measured with a Wheatstone bridge, the measured resistance change is converted into a corresponding intracranial pressure, and the corresponding intracranial pressure in the form of electrical signal is sent to an external processor for processing.

5. A non-invasive apparatus for sensing intracranial pressure comprising:
an annular flexible headband member comprising a crossing arcuate rail member and a hook and loop fastener having a fastener portion at one end of the headband member and a cooperating fastener portion at the other end of the headband member; and
a monitoring assembly comprising an inverted T-shaped, hollow seat slidably mounted on the rail member, a fastener driven through two ends of the seat for retaining the seat on the rail member, a receptacle extending downward from the seat opposing the fastener, a biasing member disposed in the receptacle, and an ultrasonic based monitor having one end secured to the biasing member and the other end moveably projecting out of the receptacle;
wherein the ultrasonic based monitor comprises a transmission module for transmitting an ultrasonic wave signal of band width toward the target, the ultrasonic wave signal being a short pulse signal, a receiving module for receiving a signal reflecting from the target, and a sending module for sending the reflecting signal representing a measured intracranial pressure to an external processor for processing.
